(19) United States
(12) Patent Application Publication
(10) Pub. No.: US 2024/0261226 A1
(43) Pub. Date: Aug. 8, 2024

(54) Medicinal and Edible Dual-Purpose Composition Capable of Resisting Retinal Blue Light Damage, and Preparation Method and Application Thereof

(71) Applicant: Dalian Polytechnic University, Dalian (CN)

(72) Inventors: Mingqian Tan, Dalian (CN); Yu Li, Dalian (CN); Jiaxuan Li, Dalian (CN); Shida Wu, Dalian (CN); Wenbo Shang, Dalian (CN); Chengfu Zhou, Dalian (CN); Wentao Su, Dalian (CN); Yukun Song, Dalian (CN); Haitao Wang, Dalian (CN)

(21) Appl. No.: 18/622,350

(22) Filed: Mar. 29, 2024

Related U.S. Application Data
(63) Continuation of application No. PCT/CN2023/113044, filed on Aug. 15, 2023.

(30) Foreign Application Priority Data
Sep. 16, 2022 (CN) 202211128626.1

(51) Int. Cl. A61K 9/20; A61K 9/00; A61K 31/05; A61K 31/353; A61K 31/721; A61K 35/60; A61K 36/45; A61K 38/17; A61P 27/02

(52) U.S. Cl. CPC A61K 9/2054; A61K 9/0053; A61K 9/2095; A61K 31/05; A61K 31/353; A61K 31/721; A61K 35/60; A61K 36/45; A61K 38/1709; A61P 27/02

(57) ABSTRACT

The disclosure discloses a medicinal and edible dual-purpose composition capable of resisting retinal blue light damage, and a preparation method and application. The medicinal and edible dual-purpose composition capable of resisting retinal blue light damage disclosed by the disclosure includes the following components in parts by weight: 40-60 parts of a microencapsulated nutrient component, 1-3 parts of a sweetening agent, 10-20 parts of microcrystalline cellulose, and 0.5-2 parts of magnesium stearate. The medicinal and edible dual-purpose composition product is simple and quick in preparation. By adding the microencapsulated nutrient component, the stability and bioavailability of procyanidine, bilberry anthocyanin and lutein are remarkably increased, synergistic anti-inflammatory and anti-oxidation functions of the procyanidine, bilberry anthocyanin and lutein are fully played, the problems of retinal damage and the like caused by LED blue light irradiation can be prevented and improved, and wide application prospects and economic benefits are achieved.

### TECHNICAL FIELD

The disclosure relates to a medicinal and edible dual-purpose composition capable of resisting retinal blue light damage, and a preparation method and application thereof, belonging to the field of medicinal and edible dual-purpose technologies.

### BACKGROUND

Blue light (400-500 nm, BL) is a kind of light with a shorter wavelength and higher energy in visible light spectra. In recent years, the demand for using electronic devices such as televisions, smart phones and computers is gradually increasing, leading to an increasing exposure of the human body to blue light. The research shows that blue light irradiation can induce oxidative stress and inflammation, damage the function of retinal pigment epitheliums (RPE), reduce the cell membrane integrity, and ultimately lead to cell apoptosis. In addition, the blue light can cause degeneration and apoptosis of photoreceptors, thus leading to eye diseases such as age-related macular degeneration (AMD).

Lutein is a natural carotenoid pigment and is also a main pigment that constitutes the macular area of the retina of the human eye. Lutein has various eye protection functions, such as scavenging free radicals in the human body, preventing macular degeneration, and protecting the RPE from photooxidation. Lutein cannot be synthesized in the human body and needs to be ingested through food. However, lutein is easily degraded by environmental factors such as light, oxygen and temperature, and loses functional activity. Furthermore, the poor solubility of lutein also limits the utilization of lutein in the human body.

Traditional medicinal and edible dual-purpose compositions generally focus on supplementing energy, relaxing the body and mind, and improving taste. In recent years, with the progress of society, people are paying more and more attention to their own health problems, and the development of leisure food with healthcare functions has become a focus of social attention. Functional candies not only have the basic features of traditional candies, but also can regulate specific physiological functions of the human body, promote human health and prevent diseases, and have an outstanding development value. The patent application of the Chinese patent CN109845868A discloses a lutein ester tablet candy capable of improving visual fatigue and a preparation method thereof. This disclosure uses lutein ester, zeaxanthin, DHA and β-carotenoid as main functional raw materials, has a simple preparation method, and can relieve visual fatigue and other problems. However, the preparation process only relies on simple proportions of raw materials and auxiliary materials to directly press into tablets as finished products, but does not solve the key problems such as the solubility and bioavailability of functional raw materials, and also does not fundamentally solve the common problem of retinal damage caused by blue light.

A direct tablet pressing method does not require processing technologies such as granulation, and can directly mix main raw materials and auxiliary materials for tablet pressing. This method has the characteristics of simple and quick process steps and high production efficiency, but has higher requirements for the moisture content and components of the processed powder. At present, this method is mainly applicable to raw materials which are unstable when exposed to moisture and heat.

Therefore, in view of the above problems and the pursuit of people for functionalization of leisure foods, it is extremely important to develop a functional leisure food which has the functions of protecting vision and resisting retinal blue light damage.

### SUMMARY

In view of the shortcomings and deficiencies in the prior art, the disclosure provides a medicinal and edible dual-purpose composition capable of resisting retinal blue light damage, and a preparation method and application thereof. In the disclosure, whey protein isolate and polyphenols are used as wall materials; procyanidine and bilberry anthocyanin, which have excellent anti-oxidation function, are noncovalently cross-linked with protein, so the emulsifying properties of the protein are increased; furthermore, the functional properties of a core material lutein can be well maintained; an obtained product has a higher embedding rate and a stable structure; an operation method is stable, efficient and pollution-free; and the bioavailability of lutein in the human body after ingestion is effectively increased to achieve excellent prevention and repair effects on blue light induced retinal damage.

The first objective of the disclosure is to provide a medicinal and edible dual-purpose composition capable of resisting retinal blue light damage. The medicinal and edible dual-purpose composition is prepared from the following raw materials in parts by weight: 40-60 parts of a microencapsulated nutrient component, 1-3 parts of a sweetening agent, 10-20 parts of microcrystalline cellulose, and 0.5-2 parts of magnesium stearate.

In an implementation, the microencapsulated nutrient component includes whey protein isolate, procyanidine, bilberry anthocyanin, deep sea fish oil, lutein, and maltodextrin.

In an implementation, the sweetening agent includes one or more of sucralose, aspartame, sucralose, and xylitol.

In an implementation, a preparation method of the microencapsulated nutrient component includes the following steps:
S1: dissolving the lutein in the deep sea fish oil to serve as an oil phase;
S2: dissolving the whey protein isolate, the procyanidine, the bilberry anthocyanin and the maltodextrin in water, and stirring and mixing the components at room temperature to serve as an aqueous phase;
S3: mixing the oil phase obtained in step S1 with the aqueous phase obtained in step S2 in a volume ratio of 1:19-3:17, and performing shearing emulsification and homogenization to obtain a nano-emulsion; and
S4: removing moisture from the nano-emulsion obtained in step S3 by a spray drying method to obtain the microencapsulated nutrient component.

In an implementation, in step S1, a mass volume ratio of the lutein to the deep sea fish oil is (0.01-0.1):1 g/ml.

In an implementation, in step S2, every 95 ml of the aqueous phase solution contains 2-7 g of whey protein isolate, 2-7 g of procyanidine, 0.1-1 g of bilberry anthocyanin, and 5-20 g of maltodextrin, and the mixing time is 3-5 h.

In an implementation, in step S3, the shearing is performed by a high-speed disperser, the rotational speed is 10000-12000 rpm, and the time is 1-2 min.

In an implementation, the homogenization is performed by a high-pressure homogenizer, the pressure is 400-600 bar, and the time is 1-2 min.

In an implementation, in step S4, spray drying conditions are as follows: the inlet temperature is 130-150° C., and the rotational speed of a peristaltic pump is 8-10 R/min.

In an implementation, the medicinal and edible dual-purpose composition is prepared from the following raw materials in parts by weight: 40-60 parts of a microencapsulated nutrient component, 1-3 parts of a sweetening agent, 10-20 parts of microcrystalline cellulose, and 0.5-2 parts of magnesium stearate. The microencapsulated nutrient component includes whey protein isolate, procyanidine, bilberry anthocyanin, deep sea fish oil, lutein, and maltodextrin.

A preparation method of the microencapsulated nutrient component includes the following steps:
S1: dissolving the lutein in the deep sea fish oil to serve as an oil phase;
S2: dissolving the whey protein isolate, the procyanidine, the bilberry anthocyanin and the maltodextrin in water, and stirring and mixing the components at room temperature to serve as an aqueous phase;
S3: mixing the oil phase obtained in step S1 with the aqueous phase obtained in step S2 in a volume ratio of 1:19-3:17, and performing shearing emulsification and homogenization to obtain a nano-emulsion; and
S4: removing moisture from the nano-emulsion obtained in step S3 by a spray drying method to obtain the microencapsulated nutrient component.

In step S1, a mass volume ratio of the lutein to the deep sea fish oil is (0.01-0.1):1 g/ml.

In step S2, every 95 ml of the aqueous phase solution contains 2-7 g of whey protein isolate, 2-7 g of procyanidine, 0.1-1 g of bilberry anthocyanin, and 5-20 g of maltodextrin.

In step S3, the rotational speed of the shearing emulsification is 10000-12000 rpm, and the time is 1-2 min. In step S3, the pressure for the homogenization is 400-600 bar, and the time is 1-2 min.

Another objective of the disclosure is to provide a preparation method of a medicinal and edible dual-purpose composition capable of resisting retinal blue light damage. The method includes: mixing a microencapsulated nutrient component and a sweetening agent, and turning and stirring the mixture for 10-30 min for uniform mixing; then, adding microcrystalline cellulose, and turning and stirring the mixture continuously for 10-20 min; and adding magnesium stearate, turning and stirring the mixture for 5-10 min, and then, directly pressing the mixture with a tablet press into tablets to obtain the medicinal and edible dual-purpose composition capable of resisting retinal blue light damage.

A third objective of the disclosure is to provide application of the above medicinal and edible dual-purpose composition in preparation of drugs capable of resisting retinal blue light damage.

The preparation method of the disclosure is simple and quick, no chemical reagents need to be added during the processing, resulting in green and healthy products; and the obtained microencapsulated nutrient component has uniform size distribution and good dispersibility, and can increase the stability of effective components such as lutein to improve the water solubility thereof, thus increasing the oral bioavailability.

In the medicinal and edible dual-purpose composition prepared by the disclosure, the procyanidine and the bilberry anthocyanin are noncovalently combined with the whey protein isolate to form a wall material to fully play synergistic effects of plant polyphenols and the lutein. The medicinal and edible dual-purpose composition has excellent anti-oxidation and anti-inflammatory effects and can effectively alleviate and prevent symptoms of retinal damage caused by LED blue light irradiation, and the product has no side effects.

### BRIEF DESCRIPTION OF FIGURES

FIG. 1A shows a scanning electron microscope of a microencapsulated nutrient component in Example 1 of the disclosure and FIG. 1B is an enlarged diagram thereof;

FIG. 2 shows the size distribution and polydispersity index (PDI) of the microencapsulated nutrient component in Example 1 of the disclosure;

FIG. 3 shows a comparison diagram of products prepared in Example 1 and Comparative Example 1 of the disclosure on RAW 264.7 macrophage cell viability;

FIG. 4 shows comparison diagrams of products prepared in Example 1 and Comparative Example 1 of the disclosure on content regulation change of tumor necrosis factor-α (TNF-α), interleukin-6 (IL-6), interleukin-1β (IL-1β) and interleukin-10 (IL-10) in an RAW 264.7 cell inflammation model;

FIG. 5 shows a comparison diagram of products prepared in Example 1 and Comparative Example 1 of the disclosure on content regulation change of total antioxidant capacity (T-AOC) of RAW 264.7 cells;

FIG. 6 shows a comparison diagram of products prepared in Example 1 and Comparative Example 1 of the disclosure on content regulation change of glutathione (GSH) in serum of each group of mice;

FIG. 7 shows a comparison diagram of products prepared in Example 1 and Comparative Example 1 of the disclosure on content regulation change of malondialdehyde (MDA) in serum of each group of mice;

FIG. 8 shows a comparison diagram of products prepared in Example 1 and Comparative Example 1 of the disclosure on content regulation change of superoxide dismutase (SOD) in serum of each group of mice; and

FIG. 9A shows comparison diagrams of products prepared in Example 1 and Comparative Example 1 of the disclosure on H&E staining change of retinal tissues in each group of mice and FIG. 9B shows measurement results.

### DETAILED DESCRIPTION

The disclosure is further described in detail below with reference to specific examples, but the implementations of the disclosure are not limited to these examples.

In the examples, lutein (purity>75%) was purchased from Shanghai Macklin Biochemical Technology Co., Ltd.;
procyanidine (purity>95%) was purchased from Tianjin Jianfeng Natural Product Research and Development Co., Ltd.;
bilberry anthocyanin (anthocyanin purity>36%, cyanidin purity>25%) was purchased from Xi'an Best Biological Technology Co., Ltd.;
whey protein isolate (purity of 80%) was purchased from Yuanye Bio-Technology Co., Ltd.;
maltodextrin (purity of 99%) was purchased from Beijing Solarbio Science & Technology Co., Ltd.;
microcrystalline cellulose (food grade) was purchased from Qingdao Wanyuan Mountain Biotech Co., Ltd.; and
deep sea fish oil was purchased from Piping Rock International Limited in the United States.

### Example 1

A preparation method of a medicinal and edible dual-purpose composition capable of resisting retinal blue light damage specifically includes the following steps:
(1) 0.15 g of lutein was dissolved in 5 mL of deep sea fish oil to serve as an oil phase;
(2) 3 g of whey protein isolate, 2.7 g of procyanidine, 0.3 g of bilberry anthocyanin and 14 g of maltodextrin were dissolved in 95 ml of deionized water and fully stirred and mixed for 3 h at room temperature to serve as an aqueous phase;
(3) the oil phase obtained in step (1) and the aqueous phase obtained in step (2) were mixed in a volume ratio of 1:19 (v/v, ml/ml), shearing emulsification was performed by a high-speed disperser at 12000 rpm for 2 min, and an emulsion obtained by emulsification was homogenized by a high-pressure homogenizer at 500 bar for 2 min to obtain a nano-emulsion;
(4) moisture was removed from the nano-emulsion obtained in step (3) by spray drying under the drying conditions that the inlet temperature was 150° C. and the rotational speed of a peristaltic pump was 10 R/min to obtain a microencapsulated nutrient component;
(5) measured by 1 g as one part: 50 parts of the microencapsulated nutrient component prepared in step (4) and 2 parts of sucralose were weighed and turned and stirred for 20 min for uniform mixing; then, 20 parts of microcrystalline cellulose was added and turned and stirred continuously for 10 min; then, 1 part of magnesium stearate was added and turned and stirred for 5 min for uniform mixing to obtain a mixture; and
(6) the mixture obtained in step (5) was directly pressed into tablets to obtain the medicinal and edible dual-purpose composition capable of resisting retinal blue light damage.

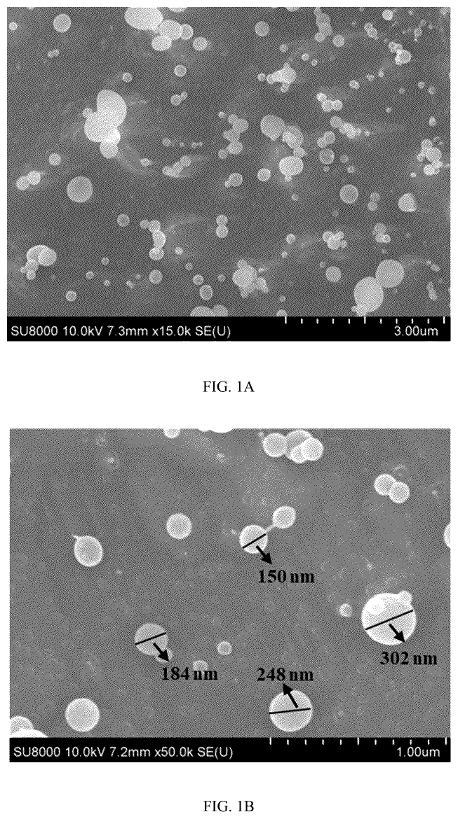

By noncovalently combining the whey protein isolate and plant polyphenols, the emulsifying capacity of the protein was enhanced, and the procyanidine and the bilberry anthocyanin were stabilized; then, by homogenization and emulsification, the lutein was successfully loaded by means of spray drying to achieve protective effects on three active substances; and the finally obtained microencapsulated nutrient component had a nano-scale spherical structure, the structure was stable, and the particle size was 150-302 nm. A scanning electron microscope of the microencapsulated nutrient component and an enlarged diagram thereof are shown in FIG. 1A-1B.

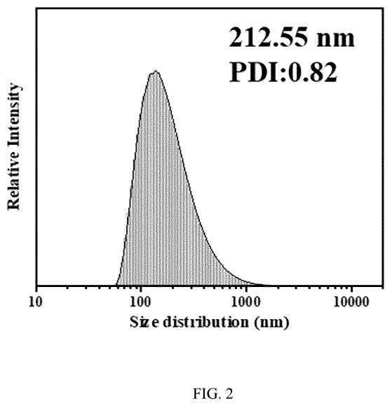

A particle size distribution diagram and a dispersion index of the microencapsulated nutrient component prepared in this example are shown in FIG. 2. Through dynamic light scattering measurement, it can be known that an average size of the obtained component was 212.55 nm, and a dispersion coefficient PDI was 0.82. It indicates that the surface active material whey protein isolate can be covalently combined with the plant polyphenols to serve as an excellent “wall material” to prevent emulsion droplets from directly aggregating with each other, so the microencapsulated nutrient component has excellent stability.

### Example 2

A preparation method of a medicinal and edible dual-purpose composition capable of resisting retinal blue light damage specifically includes the following steps:
(1) 0.20 g of lutein was dissolved in 5 mL of deep sea fish oil to serve as an oil phase;
(2) 4 g of whey protein isolate, 3.6 g of procyanidine, 0.4 g of bilberry anthocyanin and 12 g of maltodextrin were dissolved in 95 mL of deionized water and fully stirred and mixed for 4 h at room temperature to serve as an aqueous phase;
(3) the oil phase obtained in step (1) and the aqueous phase obtained in step (2) were mixed in a volume ratio of 1:19 (v/v, ml/ml), shearing emulsification was performed by a high-speed disperser at 11000 rpm for 1.5 min, and an emulsion obtained by emulsification was homogenized by a high-pressure homogenizer at 400 bar for 1.5 min to obtain a nano-emulsion;
(4) moisture was removed from the nano-emulsion obtained in step (3) by spray drying under the drying conditions that the inlet temperature was 140° C. and the rotational speed of a peristaltic pump was 9 R/min to obtain a microencapsulated nutrient component;
(5) measured by 1 g as one part: 40 parts of the microencapsulated nutrient component prepared in step (4), 1 part of aspartame, 1 part of sucralose and 1 part of xylitol were weighed and turned and stirred for 30 min for uniform mixing; then, 15 parts of microcrystalline cellulose was added and turned and stirred continuously for 15 min; then, 1.5 parts of magnesium stearate was added and turned and stirred for 5 min for uniform mixing to obtain a mixture; and
(6) the mixture obtained in step (5) was directly pressed into tablets to obtain the medicinal and edible dual-purpose composition capable of resisting retinal blue light damage.

### Example 3

A preparation method of a medicinal and edible dual-purpose composition capable of resisting retinal blue light damage specifically includes the following steps:
(1) 0.25 g of lutein was dissolved in 5 mL of deep sea fish oil to serve as an oil phase;
(2) 5 g of whey protein isolate, 4.5 g of procyanidine, 0.5 g of bilberry anthocyanin and 10 g of maltodextrin were dissolved in 95 mL of deionized water and fully stirred and mixed for 5 h at room temperature to serve as an aqueous phase;
(3) the oil phase obtained in step (1) and the aqueous phase obtained in step (2) were mixed in a volume ratio of 1:19 (v/v, ml/ml), shearing emulsification was performed by a high-speed disperser at 10000 rpm for 1 min, and an emulsion obtained by emulsification was homogenized by a high-pressure homogenizer at 600 bar for 2 min to obtain a nano-emulsion;
(4) moisture was removed from the nano-emulsion obtained in step (3) by spray drying under the drying conditions that the inlet temperature was 130° C. and the rotational speed of a peristaltic pump was 8 R/min to obtain a microencapsulated nutrient component;
(5) measured by 1 g as one part: 40 parts of the microencapsulated nutrient component prepared in step (4) and 1 part of aspartame were weighed and turned and stirred for 10 min for uniform mixing; then, 10 parts of microcrystalline cellulose was added and turned and stirred continuously for 10 min; then, 2 parts of magnesium stearate was added and turned and stirred for 10 min for uniform mixing to obtain a mixture; and
(6) the mixture obtained in step (5) was directly pressed into tablets to obtain the medicinal and edible dual-purpose composition capable of resisting retinal blue light damage.

### Comparative Example 1

Commercially available product: bilberry lutein ester β-carotene soft capsule produced by By-Health Co., Ltd., patent publication No.: CN113475714A. Main raw materials of the product include: bilberry extract, lutein ester, natural ß-carotenoid oil, soybean oil, beeswax, natural vitamin E, gelatin, purified water, glycerol, and caramel pigment.

### Performance Result Measurement

The medicinal and edible dual-purpose composition capable of resisting retinal blue light damage prepared in Example 1 and the product in Comparative Example 1 were subjected to the following cell and animal tests.

A medium involved was a high glucose medium (dulbecco's modified eagle medium (DMEM)) containing 10% of fetal bovine serum and 1% of commercialized antibiotics (penicillin and streptomycin mixed solution; SV30010; Beijing Biocity Co., Ltd.) by volume.

TNF-α, IL-6, IL-1β, IL-10, T-AOC and GSH detection kits were all purchased from Nanjing Jiancheng Bioengineering Institute.

1. Influence of a medicinal and edible dual-purpose composition with a function of resisting blue light damage on cell viability

Test cells were RAW 264.7 mouse-derived mononuclear macrophages. A cell experiment was specifically as follows: 100 μL of cell suspension was inoculated in a 96-well plate (cell density was 1×10⁴ cells/mL), and cultured in a 5% CO₂ cell incubator at 37° C. for 24 h; after complete cell adherence, the medium was discarded, and then, 100 μL of product solutions (effective lutein concentration in the solutions was 0, 40, 50, 60, 80 and 100 μg/mL respectively) in Example 1 and Comparative Example 1, diluted with a new medium and having different concentration, were added respectively; after standing for incubation for 24 h, 20 μL of MTT with a concentration of 5 μg/mL was added for further incubation for 4 h; and after a supernatant was removed, 150 μL of dimethyl sulfoxide (DMSO) was added and fully shaken for 15 min, and then, the absorbance was measured at 570 nm through an enzyme-linked immunosorbent assay (ELISA) reader to detect the cell viability.

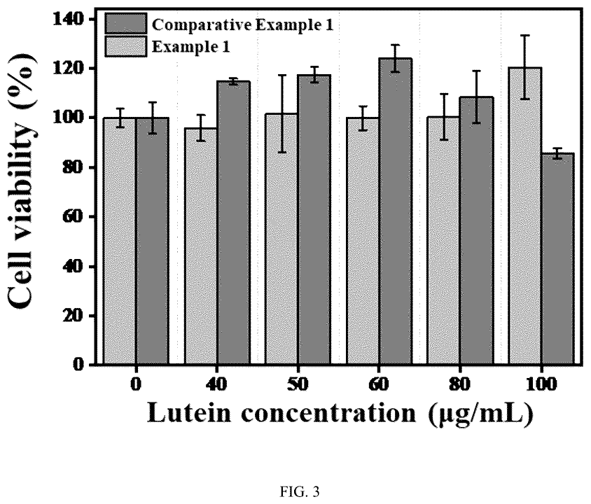

FIG. 3 shows a comparison diagram of Example 1 and Comparative Example 1 on RAW 264.7 macrophage viability. As shown in the figure, according to an MTT assay of the RAW 264.7 cell viability, at a low concentration, Example 1 and Comparative Example 1 both show that the cell viability is in direct proportional to an administration concentration. However, the bilberry lutein β-carotenoid soft capsule produced by By-Health Co., Ltd., used in Comparative Example 1, has a significant decrease in cell survival rate at a high concentration of 100 μg/mL, and it indicates an obvious damage effect on cells. The sample prepared in Example 1 has a much higher effect on cell proliferation than Comparative Example 1 (P≤0.05) at the concentration of 100 μg/mL. The above cell experiment results indicate that the product prepared in this example is non-toxic to the entire concentration range of cells and can be better absorbed by cells at higher concentration, thus increasing the cell proliferation capacity and being beneficial for high concentration ingestion.

2. Evaluation of an anti-inflammatory capacity of a medicinal and edible dual-purpose composition with a function of resisting blue light damage at a cellular level

Test cells were RAW 264.7 mouse-derived mononuclear macrophages. A cell experiment was specifically as follows: 2 mL of cell suspension was inoculated in a 6-well plate (cell density was 1×10⁶ cells/mL), and cultured in a 5% CO₂ cell incubator at 37° C. for 24 h; after complete adherence, a new medium was replaced for a blank group, a new medium solution containing a test sample (lutein concentration was 100 μg/mL) was replaced for a test group, and furthermore, 1 ng/ml of LPS was added for co-incubation (no test sample was added in a control group); and a cell suspension was collected after 24 h, a supernatant was taken after centrifugation, and the contents of correlation factors TNF-α, IL-6, IL-1β and IL-10 were measured and verified.

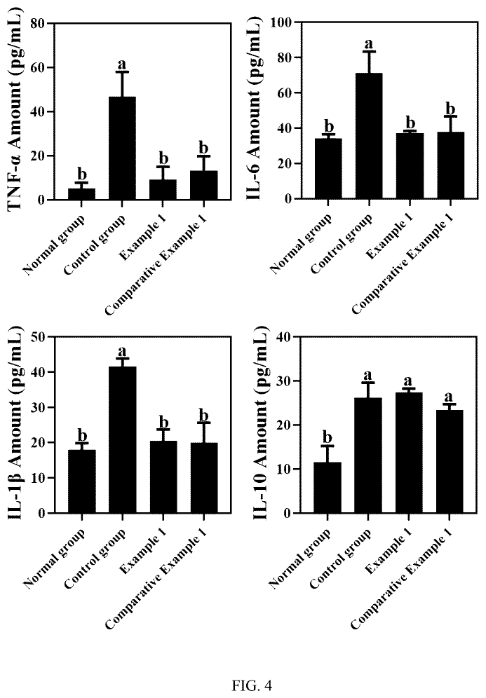

FIG. 4 shows comparison diagrams on content change of inflammation-associated factors in an LPS-induced RAW 264.7 cell inflammation model. As shown in the figure, compared to the blank group, after RAW 264.7 cells were stimulated by LPS, the levels of pro-inflammatory factors (IL-6, IL-1β) in the control group were significantly increased (P≤0.05), and the level of an anti-inflammatory factor (IL-10) was reduced. After processing by two products, compared to the control group, the TNF-α and IL-1β levels of cells were significantly reduced (P≤0.05) and restored to approach the blank group.

The level of the pro-inflammatory factor IL-6 in Example 1 Group was slightly lower than that in Comparative Example 1 Group, and the level of the anti-inflammatory cell factor (IL-10) was significantly higher than that in Comparative Example 1 Group. The above results indicate that the medicinal and edible dual-purpose composition with a function of resisting blue light damage, prepared in Example 1, has an excellent anti-inflammatory effect and can significantly relieve the degree of the cell inflammation caused by LPS stimulation.

TABLE 1
Release amounts of inflammatory factors in cell inflammation
model in Example 1 and Comparative Example 1

| Test | TNF-α | IL-6 | IL-1β | IL-10 |
|---|---|---|---|---|
| Sample | (pg/mL) | (pg/mL) | (pg/mL) | (pg/mL) |
| Normal group | 5.07 ± 2.62 | 34.24 ± 2.26 | 17.91 ± 1.85 | 11.47 ± 3.70 |
| Control group | 46.86 ± 11.08 | 71.32 ± 12.04 | 41.70 ± 2.14 | 26.23 ± 3.38 |
| Example 1 | 9.09 ± 5.80 | 37.05 ± 1.33 | 20.42 ± 3.24 | 27.39 ± 0.90 |
| Comparative | 13.25 ± 6.47 | 37.89 ± 8.71 | 19.90 ± 5.73 | 23.39 ± 1.33 |
| Example 1 | | | | |

The group without any treatment was regarded as normal group (blank group).

The group which treated only with LPS or H₂O₂ was regarded as control group.

3. Evaluation of an antioxidant capacity of a medicinal and edible dual-purpose composition with a function of resisting blue light damage at a cellular level

Test cells were RAW 264.7 mouse-derived mononuclear macrophages. A cell experiment was specifically as follows: 100 μL of cell suspension was inoculated in a 96-well plate (cell density was 1×10⁴ cells/mL), and cultured in a 5% CO₂ cell incubator at 37° C. for 24 h; after complete cell adherence, a new medium was replaced for a blank group, a new medium solution containing a test sample (lutein concentration was 100 μg/mL) was replaced for a test group, and furthermore, 500 μmol/L of H₂O₂ was added for co-incubation (no test sample was added in a control group); and after 12 h, the cell viability was measured by an MTT assay.

2 mL of cell suspension was inoculated in a 6-well plate (cell density was 1×10⁶ cells/mL), and cultured in a 5% CO₂ cell incubator at 37° C. for 24 h; after complete cell adherence, a new medium was replaced for a blank group, a new medium solution containing a test sample (lutein concentration was 100 μg/mL) was replaced for a test group, and furthermore, 500 μmol/L of H₂O₂ was added for co-incubation (no test sample was added in a control group); and after 12 h, the medium was discarded, the inside of the well plate was cleaned by PBS, then cells were scraped by a cell scraper, a cell fragmentation solution was obtained by an ultrasonic fragmentation method, and the T-AOC and GSH content were measured.

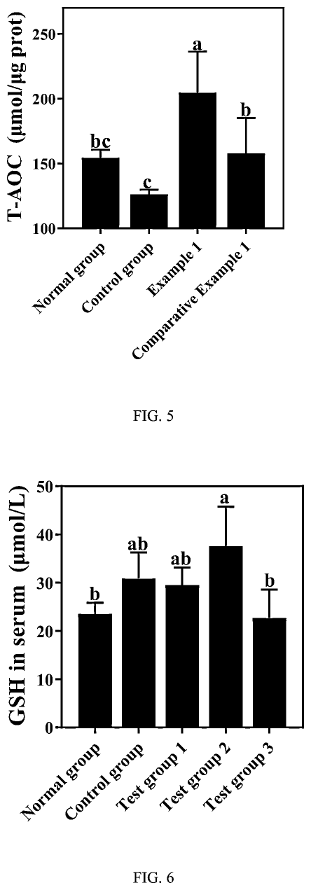

The T-AOC reflects the capacity of cells for scavenging free radicals and inhibiting lipid peroxidation. As shown in FIG. 5, after processing by Example 1 Group, the T-AOC level of cells was significantly higher than that of the product in Comparative Example 1 (P≤0.05).

In conclusion, the medicinal and edible dual-purpose composition with a function of resisting blue light damage, prepared in Example 1, has an excellent in vitro antioxidant effect, and can significantly improve the total antioxidant capacity and free radical scavenging capacity of cells under damage conditions.

4. Influence of a medicinal and edible dual-purpose composition with a function of resisting blue light damage on retinas of LED blue light damage model mice

Balb/c 5-week-old male mice (purchased from Liaoning Changsheng Biotechnology Co., Ltd.) were selected. All animal experiments were performed in accordance with the UK's Animal (Scientific Procedures) Act 1986 and relevant guidelines as well as EU Directive 2010/63/EU, and the guidelines and animal protocol (Dlpu2020024) have been approved by the Ethics Committee of Dalian Polytechnic University.

Mice were fed in standard cages and fed cyclically in the light and dark for 12:12 h at 25±1° C. After one week of adapting to freely eating and drinking water, the mice were randomly divided into 5 groups (6 mice in each group).

Blank group and control group: Mice were fed with 200 μl of purified water by gavage every day.

Test group 1: Mice were fed with 200 μL of an aqueous solution of the product in Example 1 by gavage every day (100 mg/kg mouse body weight).

Test group 2: Mice were fed with 200 μL of an aqueous solution of the product in Example 1 by gavage every day (50 mg/kg mouse body weight).

Test group 3: Mice were fed with 200 μL of an aqueous solution of the product in Comparative Example 1 by gavage every day (50 mg/kg mouse body weight).

After continuous gavage for 5 days, except for the blank group, all other groups of mice were left in a dark room for 1 h after gavage and then exposed to a blue light strip with the light intensity of 7000 Ix for irradiation for 1 h. After 5 days, the duration of blue light irradiation was increased to 2 h. After 9 days of high-duration irradiation, blood samples were obtained from the eyeballs of the mice and then centrifuged, and serum was extracted to measure the contents of GSH, MDA and SOD. The removed eyeballs were fixed in a Davidson's solution fixative (a volume ratio of 10% neutral buffered formaldehyde to 95% ethanol to glacial acetic acid to distilled water was 1:3:1:3, Liaoning Jijia Biotechnology Co., Ltd.). Subsequently, the eyeballs were taken out for paraffin embedding, slicing, dehydration, fixation, staining with hematoxylin and eosin (H&E), and retinal tissues at a distance of 600 to 900 mm from optic nerves were measured at an interval of 60 mm. The thicknesses of an outer nuclear layer (ONL), an inner nuclear layer (INL), a photosensitive layer (PL) and the entire retina were computed by Image J software.

The comparison of changes in GSH content in serum of mice is shown in FIG. 6. GSH is considered as one of the important factors in maintaining redox balance in organisms. After gavage therapy, the GSH content in the serum in the test group 2 was the highest, and there was a significant difference (P≤0.05) compared to the test group 3.

MDA is a lipid peroxidation product, and the level of MDA indirectly reflects the severity of free radical attacks on cells in the body. As shown in FIG. 7, after LED blue light irradiation, the MDA level in the serum of mice was significantly increased, indicating that the LED blue light can cause oxidative damage to the body. After gavage therapy, the MDA content was significantly reduced and restored to approach the level of the blank group.

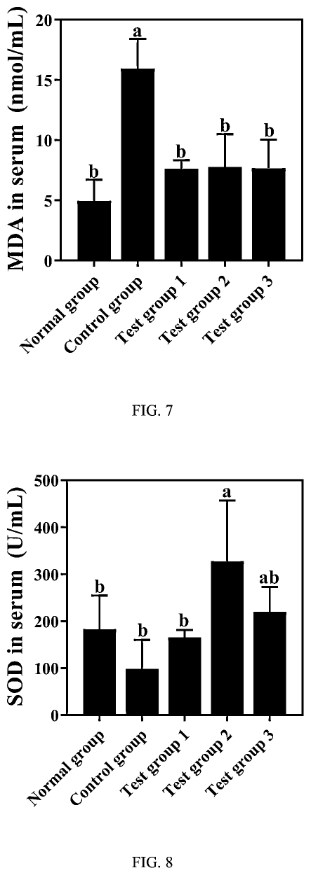

Similarly, SOD indirectly reflects the capacity of the body for scavenging oxygen free radicals. As shown in FIG. 8, compared to the control group, the SOD content in the serum of the mice in the test group 2 was significantly increased (P≤0.05). The data related to the serum of the mice shows that the product prepared in Example 1 has significant capacities for scavenging free radicals in the body, resisting oxidation, and the like.

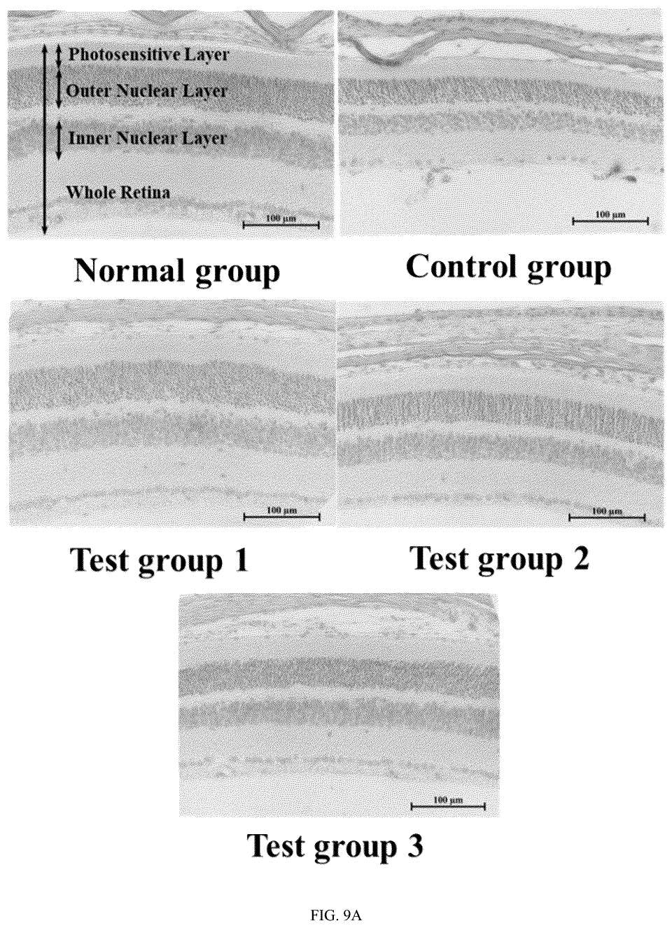

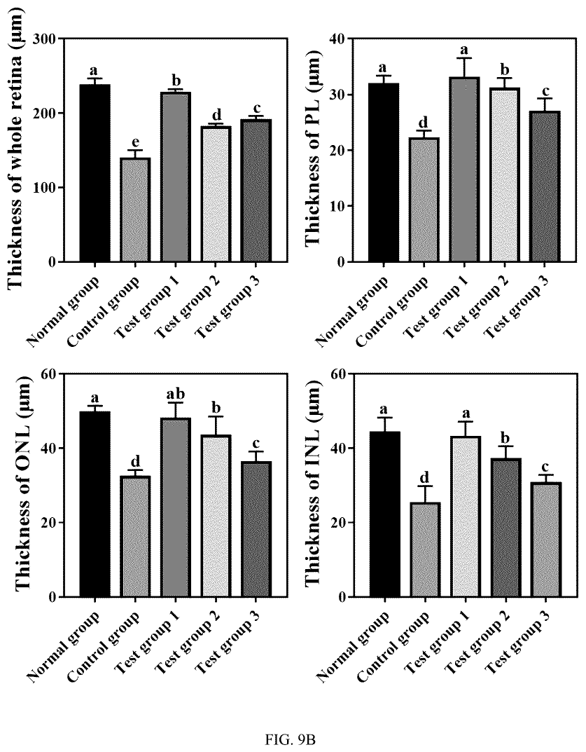

FIG. 9A-9B shows H&E staining diagrams of retinal tissue slices of mice. The measurement results show that after LED blue light irradiation, the total thickness of the retina and the thicknesses of the components of the retina were all significantly reduced. After therapy in the test group 1 and the test group 2, the thicknesses of the PL, ONL and INL and the total thickness of the retina were all significantly restored, and there were significant differences compared to the test group 3 and the control group.

The results of mouse animal experiments indicate that the medicinal and edible dual-purpose composition with a function of resisting blue light damage, prepared in Example 1, has a function of significantly and effectively resisting retinal damage caused by LED blue light irradiation, and comprehensive effects are higher than those of the two commodities in Comparative Example.

In the disclosure, the medicinal and edible dual-purpose composition with a function of resisting blue light damage is prepared by a direct tablet pressing method, production and processing methods are simple and quick, and no chemical components are added, so the composition is green and healthy. Furthermore, by a microencapsulation technology, full protection of lutein, procyanidine and bilberry anthocyanin is achieved, the stability of nutrient components is improved, and synergistic effects are played. Test results indicate that the product of the disclosure has excellent antioxidant and anti-inflammatory effects and can significantly improve the problems such as retinal damage caused by blue light stimulation, and comprehensive effects are higher than those of the commercially available chewable tablets and soft capsules in Comparative Example.

The above examples are preferred examples of the disclosure, and the disclosure is not limited to the specific conditions and details in the above examples. Any other changes, modifications, substitutions, combinations and simplifications made without departing from the spirit essence and principle of the disclosure shall be equivalent replacement modes, and shall be included in the scope of protection of the disclosure.

What is claimed is:

1. A medicinal and edible dual-purpose composition capable of resisting retinal blue light damage, wherein the medicinal and edible dual-purpose composition comprises the following raw materials in parts by weight: 40-60 parts of a microencapsulated nutrient component, 1-3 parts of a sweetening agent, 10-20 parts of microcrystalline cellulose, and 0.5-2 parts of magnesium stearate.

2. The medicinal and edible dual-purpose composition capable of resisting retinal blue light damage according to claim 1, wherein the microencapsulated nutrient component comprises whey protein isolate, procyanidine, bilberry anthocyanin, deep sea fish oil, lutein, and maltodextrin.

3. The medicinal and edible dual-purpose composition capable of resisting retinal blue light damage according to claim 1, wherein the sweetening agent comprises one or more of xylitol, sucralose, aspartame, and sorbitol.

4. The medicinal and edible dual-purpose composition capable of resisting retinal blue light damage according to claim 1, wherein a preparation method of the microencapsulated nutrient component comprises the following steps:
S1: dissolving the lutein in the deep sea fish oil to serve as an oil phase;
S2: dissolving the whey protein isolate, the procyanidine, the bilberry anthocyanin and the maltodextrin in water, and stirring and mixing the components at room temperature to serve as an aqueous phase;
S3: mixing the oil phase obtained in step S1 with the aqueous phase obtained in step S2 in a volume ratio of 1:19-3:17, and performing shearing emulsification and homogenization to obtain a nano-emulsion; and
S4: removing moisture from the nano-emulsion obtained in step S3 by a spray drying method to obtain the microencapsulated nutrient component.

5. The medicinal and edible dual-purpose composition capable of resisting retinal blue light damage according to claim 4, wherein in step S1, a mass volume ratio of the lutein to the deep sea fish oil is (0.01-0.1):1 g/ml.

6. The medicinal and edible dual-purpose composition capable of resisting retinal blue light damage according to claim 4, wherein in step S2, every 95 ml of the aqueous phase solution contains 2-7 g of whey protein isolate, 2-7 g of procyanidine, 0.1-1 g of bilberry anthocyanin, and 5-20 g of maltodextrin.

7. The medicinal and edible dual-purpose composition capable of resisting retinal blue light damage according to claim 4, wherein in step S3, the rotational speed of the shearing emulsification is 10000-12000 rpm, and the time is 1-2 minutes.

8. The medicinal and edible dual-purpose composition capable of resisting retinal blue light damage according to claim 4, wherein in step S3, the pressure for the homogenization is 400-600 bar, and the time is 1-2 minutes.

9. A preparation method of the medicinal and edible dual-purpose composition capable of resisting retinal blue light damage according to claim 1, wherein the method comprises: mixing the microencapsulated nutrient component and the sweetening agent, and turning and stirring the mixture for 10-30 minutes for uniform mixing; then, adding the microcrystalline cellulose, and turning and stirring the mixture continuously for 10-20 minutes; and adding the magnesium stearate, turning and stirring the mixture for 5-10 minutes, and then, directly pressing the mixture with a tablet press into tablets to obtain the medicinal and edible dual-purpose composition capable of resisting retinal blue light damage.

10. Application of the medicinal and edible dual-purpose composition capable of resisting retinal blue light damage according to claim 1 in preparation of drugs capable of resisting retinal blue light damage, wherein the application comprises: mixing the medicinal and edible dual-purpose composition and excipients for tablet pressing or capsule making.
